(19) United States
(12) Patent Application Publication
(10) Pub. No.: US 2023/0320654 A1
(43) Pub. Date: Oct. 12, 2023

(54) SPINAL IMPLANTS WITH ELECTRONICS CARTRIDGE AND EXTERNALIZED ANTENNA

(71) Applicant: Warsaw Orthopedic, Inc., Warsaw, IN (US)

(72) Inventors: Brad Tischendorf, Minneapolis, MN (US); Arjun Siby-Kurian, Memphis, TN (US); Newton H. Metcalf, Memphis, TN (US)

(73) Assignee: Warsaw Orthopedic, Inc., Warsaw, IN (US)

(21) Appl. No.: 18/183,484

(22) Filed: Mar. 14, 2023

Related U.S. Application Data
(63) Continuation-in-part of application No. 18/062,867, filed on Dec. 7, 2022.
Application No. 18/062,867 is a continuation-in-part of application No. 18/067,140, filed on Dec. 16, 2022.
(60) Provisional application No. 63/329,982, filed on Apr. 12, 2022.

(51) Int. Cl. A61B 5/00

(52) U.S. Cl. CPC A61B 5/4566; A61B 5/48

(57) ABSTRACT

A spinal implant includes an electronics portion with a housing defining a sealed cavity for supporting an electronics assembly and a battery therein. The spinal implant further includes at least one antenna in electrical communication with the electronics assembly and at least one sensor in electrical communication with the electronics assembly, wherein the at least one antenna is located external to the housing and is configured to transmit information received from the at least one sensor to an external device.

### CROSS-REFERENCE TO RELATED APPLICATIONS

This application claims priority to U.S. Provisional Application 63/329,982, titled SMART IMPLANT DESIGNS FOR HOUSING A POWER SOURCE, ANTENNA, GAUGES, AND MICROELECTRONICS, and filed Apr. 12, 2022. This application is also a continuation-in-part of U.S. application Ser. No. 18/062,867, titled SPINAL ROD CONNECTING COMPONENTS WITH ACTIVE SENSING CAPABILITIES, and filed Dec. 7, 2022. This application is also a continuation-in-part of U.S. application Ser. No. 18/068,140, titled SPINAL IMPLANTS WITH ACTIVE SENSING CAPABILITIES, and filed Dec. 19, 2022. The disclosures of these applications are incorporated herein by reference in their entirety.

### FIELD

The present disclosure generally relates to mechanical and electrical sensor assemblies and antenna designs for implant devices, and more particularly to spinal implant systems which may be used to treat various spinal disorders.

### BACKGROUND

Treatment of spinal disorders, such as degenerative disc disease, disc herniations, scoliosis or other curvature abnormalities, and fractures, often requires surgical treatments. For example, spinal fusion may be used to limit motion between vertebral members. As another example, implants may be used to preserve motion between vertebral members.

Surgical treatment typically involves the use of implants and longitudinal members, such as spinal rods. Implants may be disposed between two vertebral members for supporting and/or repositioning the vertebral members. Implants may also be used to facilitate a fusion process between a superior vertebra and an inferior vertebra. Longitudinal members may be attached to the exterior of two or more vertebral members to assist with the treatment of a spinal disorder. Longitudinal members may provide a stable, rigid column that helps bones to fuse, and may redirect stresses over a wider area away from a damaged or defective region. Also, rigid longitudinal members may help in spinal alignment.

Screw assemblies may be used to connect a longitudinal member to a vertebral member. A screw assembly may include a pedicle screw, hook, tulip bulb connector or other type of receiver, and a set screw, among other components. A pedicle screw can be placed in, above and/or below vertebral members that were fused, and a longitudinal member can be used to connect the pedicle screws which inhibit or control movement. A set screw can be used to secure the connection of a longitudinal member and a pedicle screw, hook, or other connector. Implants may include one or more sensors for monitoring aspects of the treatment and transmitting sensor data to an external reader. However, the configuration of an antenna may be constrained by the patient's anatomy, reducing the antenna's effectiveness. Furthermore, the tissue surrounding the implants can attenuate transmitted signals. This document describes methods and systems that are directed to addressing the problems described above, and/or other issues.

### SUMMARY

The techniques of this disclosure generally relate to spinal implants having various sensors for communicating attributes about the spinal implants, when installed in patient anatomy, to an external reader, via an externalized antenna. In an example embodiment, a spinal implant is disclosed. The spinal implant includes an electronics portion with a housing defining a sealed cavity for supporting an electronics assembly and a battery therein. The spinal implant further includes at least one antenna in electrical communication with the electronics assembly and at least one sensor in electrical communication with the electronics assembly, wherein the at least one antenna is located external to the housing and is configured to transmit information received from the at least one sensor to an external device.

Implementations of the disclosure may include one or more of the following optional features. In some examples, the housing includes a hermetically sealed, removable electronics cartridge having at least one antenna interface and the electronics portion is configured to receive the removable electronics cartridge. The removable electronics cartridge may have a threaded exterior that mates with the electronics portion. The at least one sensor may be located within the removable electronics cartridge. In some examples, the spinal implant includes a pedicle screw having a set screw, the housing includes a removable electronics cartridge having at least one antenna interface, and the set screw is configured to receive the removable electronics cartridge. In some examples, the spinal implant includes an interbody cage and the at least one antenna is wound around an exterior surface of the interbody cage. In some examples, the interbody cage includes a slot and the at least one antenna is wound around the exterior surface of the interbody cage and through the slot. In some examples, the spinal implant includes a pedicle screw and the at least one antenna is configured to flexibly extend away from the pedicle screw. The at least one sensor may include at least one temperature sensor configured to measure a temperature of a surgical site. The at least one sensor may include at least one strain gauge configured to measure a localized force experienced by the spinal implant. In some examples, the spinal implant includes an interbody cage including a graft window and the at least one sensor includes at least one impedance sensor located within the graft window and configured to measure a status of a fusion process. The at least one sensor may be chosen from the group including: accelerometer, gyroscope, strain gauge, pressure sensor, pH sensor, impedance sensor, optical sensor, and temperature sensor. The battery may be configured to be inductively recharged.

In an example embodiment, a surgical site monitoring system is disclosed. The surgical site monitoring system includes a first spinal implant and a second spinal implant. The first spinal implant includes a first electronics portion including a first housing defining a first sealed cavity for supporting a first electronics assembly and a first battery therein. The first spinal implant further includes at least one first antenna in electrical communication with the first electronics assembly and at least one first sensor in electrical communication with the first electronics assembly, wherein the at least one first antenna is located external to the first housing and is configured to transmit first information received from the at least one first sensor to an external device. The second spinal implant includes a second electronics portion including a second housing defining a second sealed cavity for supporting a second electronics assembly and a second battery therein. The second spinal implant further includes at least one second antenna in electrical communication with the second electronics assembly and at least one second sensor in electrical communication with the second electronics assembly, wherein the at least one second antenna is located external to the second housing and is configured to transmit second information received from the at least one second sensor to an external device.

Implementations of the disclosure may include one or more of the following optional features. In some examples, the first and second housings include hermetically sealed, removable electronics cartridges having at least one antenna interface and the first and second electronics portions are configured to receive the removable electronics cartridges. In some examples, the at least one first sensor and the at least one second sensor include at least one temperature sensor configured to collect temperature measurements within a patient. The at least one first sensor and the at least one second sensor may include at least one impedance sensor configured to measure a status of a fusion process. The first and second spinal implants may each include a pedicle screw or an interbody cage. In some examples, the first and second antennas are each configured to flexibly extend away from the first and second spinal implants, respectively. In some examples, at least one of the first battery and the second battery are configured to be inductively recharged.

### BRIEF DESCRIPTION OF DRAWINGS

FIG. 1 is a perspective view of an example embodiment of a pedicle screw system.

FIG. 2 is a top down view of the embodiment of FIG. 1.

FIG. 3 is a first side view of the embodiment of FIG. 1.

FIG. 4 is a second side view of the embodiment of FIG. 1.

FIG. 5 is a first exploded parts view of the embodiment of FIG. 1.

FIG. 6 is a second exploded parts view of a receiver portion and sensing components of the embodiment of FIG. 1.

FIGS. 7 and 8 are perspective views of an example embodiment of a pedicle screw system.

FIGS. 9 and 10 are perspective views of an example embodiment of a pedicle screw system.

FIG. 11 is a perspective view of an example embodiment of a spinal implant.

FIG. 12 is an exploded parts view of an example embodiment of a spinal implant.

FIG. 13 is a perspective view of an example embodiment of a spinal implant.

FIG. 14 is a perspective view of an example embodiment of a spinal implant.

FIG. 15 is a perspective view of an example embodiment of a spinal implant.

FIG. 16 illustrates an example of a surgical site monitoring system according to an embodiment.

### DETAILED DESCRIPTION

Embodiments of the present disclosure relate generally, for example, to spinal implant systems with active sensing, microelectronics, and communication abilities. Embodiments of the devices and methods are described below with reference to the Figures.

The following discussion omits or only briefly describes certain components, features and functionality related to medical implants, installation tools, and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment has the same or nearly the same functionality or characteristic, unless the context or other statements clearly indicate otherwise. The term “about” may encompass a meaning of being +/−10% of the stated value.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

The disclosed medical-implant embodiments include pedicle screws and interbody cages that are configured to provide telemetry to an external device. Telemetry data may include position/motion information, force/strain information, temperature, tissue impedance, and so forth. For example, the spinal implants may be installed during a surgical procedure such as a spinal fusion and may be configured to sense aspects of the patient's post-operative recovery, such as forces between spinal implants. The spinal implants may provide telemetry related to the surgical site, such as temperature readings from a variety of locations around the surgical site, e.g., to sense and localize an infection. The spinal implants may provide telemetry related to motion of the patient's spine, e.g., while performing a post-operative diagnostic regimen. In these cases and others, an external reader, such as the system disclosed in U.S. patent application Ser. No. 16/855,444, incorporated herein by reference in its entirety, may display or otherwise provide the telemetry to a medical professional for evaluation. The external reader may also receive telemetry from other sources such as, but not limited to, one or more wearable sensor system that are affixed to the patient. The reader device itself may include additional sensors as well.

The medical implants may include electronics, such as sensors or sensor systems which acquire the telemetry data, and transmitter (or transceiver) systems which transmit the telemetry to an external reader/receiver device. The spinal implants may also include a power source, such as a battery (rechargeable or otherwise) for powering the electronics. The transmitter system may include an antenna for radiating the telemetry signal to the reader device (and/or an intermediate relay device). However, the antenna size and/or configuration may be constrained by the patient's anatomy in various ways, reducing the antenna's effectiveness. For example, the transmitter may need to transmit the telemetry through six or more inches of tissue to reach the external reader device. This distance may be near the limit for effective telemetry. The size of the power source may also be constrained (e.g., by the patient's anatomy or for regulatory reasons, etc.), limiting the possible strength of the transmitted signal (e.g., while achieving reasonable battery lifetime or reasonable time between battery recharges).

The tissue surrounding the implant may further constrain antenna configuration. Components of a vertebral column may move with respect to one another (and to the surrounding tissue). Therefore, a rigid antenna extending away from a spinal implant may irritate or damage surrounding tissue as the implant moves with respect to the surrounding tissue. The level of irritation or damage may be related to the type of tissue as well as the construction and/or configuration of the antenna. For example, the vicinity around an interbody cage includes, among other things, the spinal cord and associated nerves extending away from the spinal cord, blood vessels of various types, as so forth. This type of tissue may be particularly sensitive to interaction with an antenna projecting from an implant and, in some cases, may be subject to irreparable harm. Other medical implants may be surrounded by less sensitive tissue, such as muscle, which may tolerate more intrusive antenna configurations.

Referring to the disclosed embodiments generally, various vertebral pedicle screw and interbody cage systems are disclosed. The components of the implant systems can be fabricated from biologically acceptable materials suitable for medical applications, including metals, synthetic polymers, ceramics and bone material and/or their composites. The components may be fabricated using bio-inert materials, such as metals, ceramics, polymers, etc. The components may also be fabricated (either entirely or at least partially) using bio-resorbable or bio-convertible materials, as appropriate. For example, the components, individually or collectively, can be fabricated from materials such as stainless steel alloys, commercially pure titanium, titanium alloys, Grade 5 titanium, super-elastic titanium alloys, cobalt-chrome alloys, super-elastic metallic alloys (e.g., Nitinol, super-elastoplastic metals, such as GUM METAL®), ceramics and composites thereof such as calcium phosphate (e.g., SKELITE™), alumina, yttria-stabilized zirconia (YSZ), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyketide, polyglycolide, polytyrosine carbonate, polycaroplaetohe, polylactic acid or polylactide and their combinations.

Various components of the implant system may be formed or constructed with material composites, including the above materials, to achieve various desired characteristics such as strength, rigidity, elasticity, compliance, biomechanical performance, durability and radiolucency or imaging preference. The components of the present implant system, individually or collectively, may also be fabricated from a heterogeneous material such as a combination of two or more of the above-described materials. The components of the implant system may be monolithically formed, integrally connected or include fastening elements and/or instruments, as described herein. The components of the implant system may be formed using a variety of subtractive and additive manufacturing techniques, including, but not limited to machining, milling, extruding, molding, 3D-printing, sintering, coating, vapor deposition, and laser/beam melting.

Furthermore, various components of the implant system may be coated or treated with a variety of additives or coatings to improve biocompatibility, bone growth promotion or other features. Various embodiments and components may be coated with a ceramic, titanium, and/or other biocompatible material to provide surface texturing at (a) the macro scale, (b) the micro scale, and/or (c) the nano scale, for example. Similarly, components may undergo a subtractive manufacturing process such as, for example, grit blasting and acid etching, providing for surface texturing configured to facilitate osseointegration and cellular attachment and osteoblast maturation. Example surface texturing of additive and subtractive manufacturing processes may include (a) macro-scale structural features having a maximum peak-to-valley height of about 40 microns to about 500 microns, (b) micro-scale structural features having a maximum peak-to-valley height of about 2 microns to about 40 microns, and/or (c) nano-scale structural features having a maximum peak-to-valley height of about 0.05 microns to about 5 microns. In various embodiments, the three types of structural features may be overlapping with one another. Additionally, such surface texturing may be applied to any surface, e.g., both external exposed facing surfaces of components and internal non exposed surfaces of components. Further discussion regarding relevant surface texturing and coatings is described in, for example, U.S. Pat. No. 11,096,796, titled Interbody spinal implant having a roughened surface topography on one or more internal surfaces, and filed on Mar. 4, 2013—the entire disclosure of which is incorporated herein by reference in its entirety. Accordingly, it shall be understood that any of the described coating and texturing processes of U.S. Pat. No. 11,096,796, may be applied to any component of the various embodiments disclosed herein, e.g., the exposed surfaces and internal surfaces. Another example technique for manufacturing an orthopedic implant having surfaces with osteoinducting roughness features including micro-scale structures and nano-scale structures is disclosed in U.S. Pat. No. 10,821,000, the entire contents of which are incorporated herein by reference. Additionally, an example of a commercially available product may be the Adaptix™ Interbody System sold by Medtronic Spine and comprising a titanium cage made with Titan nanoLOCK™.

The disclosed implant systems may be employed, for example, with a minimally invasive procedure, including percutaneous techniques, mini-open and open surgical techniques to deliver and introduce instrumentation and/or one or more spinal implants at a surgical site within a body of a patient, for example, a section of a spine. In some embodiments, the implant system may be employed with surgical procedures, as described herein, and/or, for example, corpectomy, discectomy, fusion and/or fixation treatments that employ spinal implants to restore the mechanical support function of vertebrae. In some embodiments, the implant system may be employed with surgical approaches, including but not limited to: anterior lumbar interbody fusion (ALIF), direct lateral interbody fusion (DLIF), oblique lateral lumbar interbody fusion (OLLIF), oblique lateral interbody fusion (OLIF), transforaminal lumbar Interbody fusion (TLIF), posterior lumbar Interbody fusion (PLIF), various types of posterior or anterior fusion procedures, and any fusion procedure in any portion of the spinal column (sacral, lumbar, thoracic, and cervical).

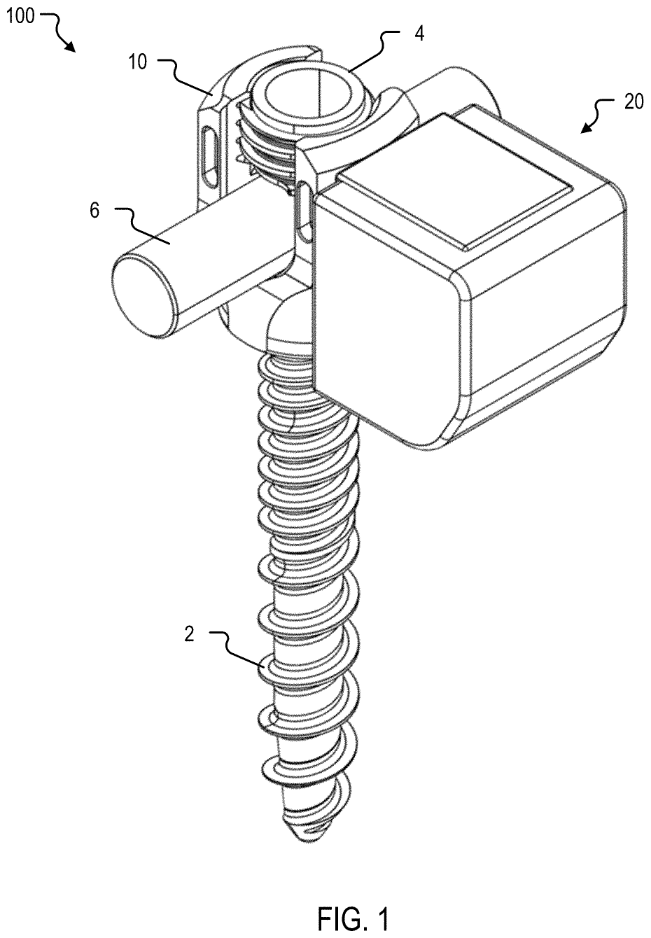

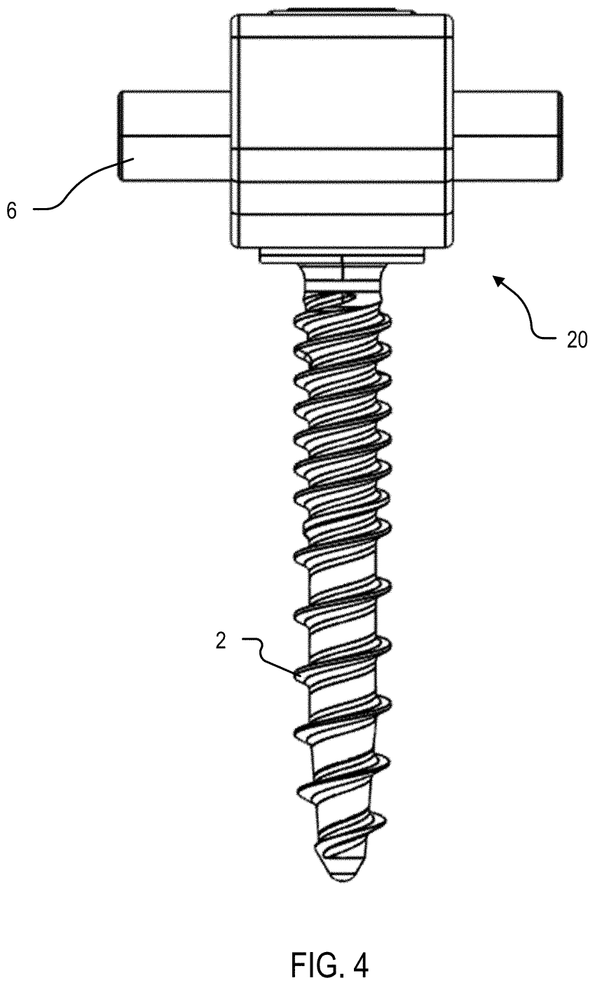

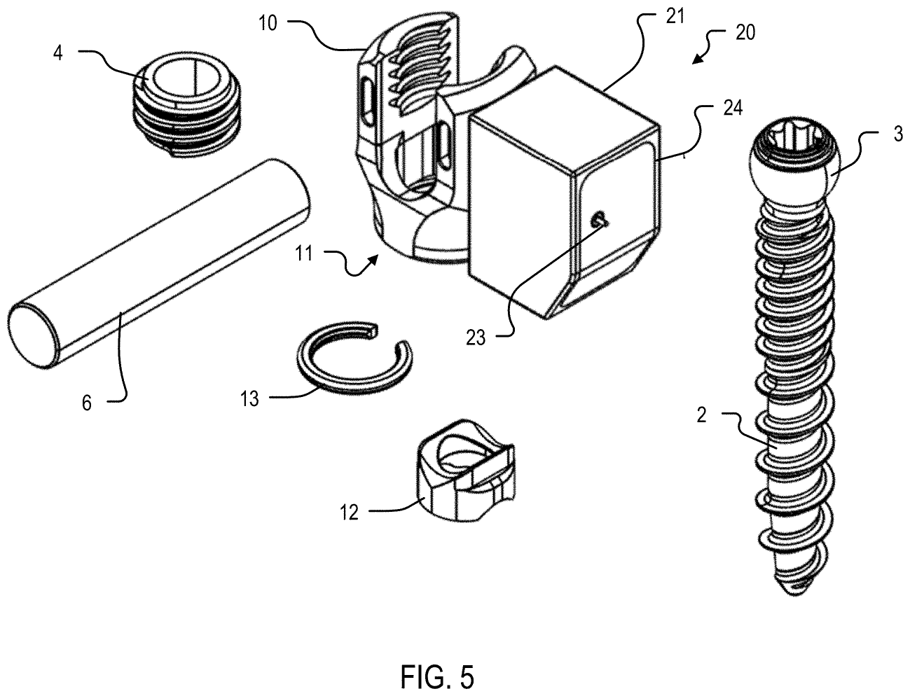

FIGS. 1-10 illustrate an example digital pedicle screw system 100 with active sensing ability. As illustrated in FIG. 1, system 100 may include a pedicle screw 2 and a receiver 10 having a side portion 20 for supporting various electronic components and sensors as will be explained in further detail below. The pedicle screw 2 may have a thread pitch extending along a length thereof for implanting and securing the pedicle screw 2 into patient anatomy, e.g., a vertebral body. As seen best in FIG. 5, the pedicle screw 2 may include a head portion 3 that may couple to the receiver 10 in a lower cavity 11. In various embodiments, a lower cavity 11 of receiver 10 may include at least one annular groove for supporting a deformable annular ring or c-ring 13 that captures the head 3 of pedicle screw 2. In this way, receiver 10 may be popped on to the head 3 of a pedicle screw 2 simply by pressing down on receiver 10 as would be understood by a person of ordinary skill in the art.

In various embodiments, the lower cavity 11 and head 3 may be configured to enable coupling of receiver 10 in a multitude of angled orientations with respect to the extension direction of pedicle screw 2. For example, receiver 10 may be configured as a multiaxial receiver. In other embodiments, receiver 10 may be configured as a monoaxial receiver. In various embodiments a saddle 12 may be disposed within the lower cavity 11 of receiver 10 to support a longitudinal rod 6 disposed in the U-shaped cavity of receiver 10. As seen best in FIG. 1, a set screw 4 may engage to threads of each respective arm of the U-shaped cavity of receiver 10. When sufficiently tightened, set screw 4 may immobilize and/or secure the longitudinal rod 6 within the U-shaped cavity of receiver 10.

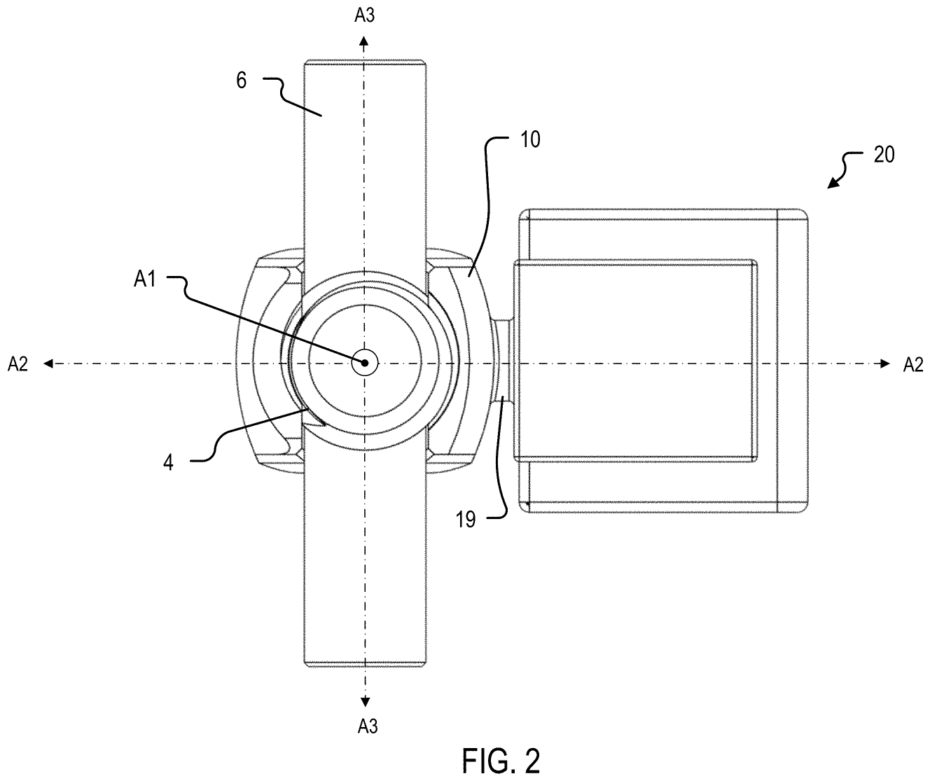

Referring to FIGS. 2-4 generally, receiver 10 may be coupled to side portion 20 via a beam portion 19. In various embodiments, receiver 10 and side portion 20 may be monolithically formed as a single piece or receiver 10 and side portion 20 may be separable pieces that are connected together. In the example embodiment, receiver 10 and side portion 20 are monolithically formed and/or integrally formed together. For example, the receiver 10 is integrally formed with the side portion 20 and they are connected via beam portion 19. This arrangement may have the advantage of facilitating the transfer of stress and strain between the receiver 10 and side portion 20 as will be explained in further detail below.

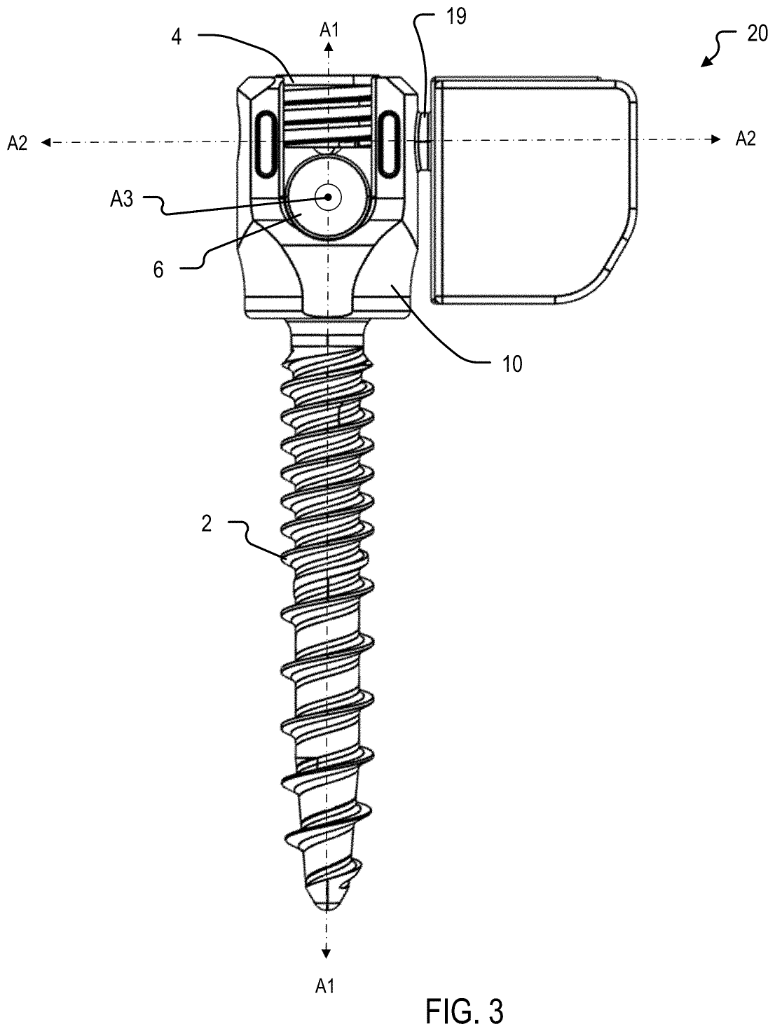

With respect to a normal viewing angle shown in FIG. 3, the receiver 10 and side portion 20 may extend in a vertical direction parallel to axis A1 (a longitudinal direction), in a first horizontal direction parallel to axis A2 (a first widthwise direction), and in a second horizontal direction parallel to axis A3 (a second widthwise direction). For ease of explanation, the particular location of the Axes A1, A2, and A3 are illustrated as being centered with respect to various components of interest of system 100. For example, axis A1 may be centered in the vertical direction with respect to the U-shaped cavity of receiver 10 and define a rotation axis of set screw 4. Axis A2 may be centered in the first widthwise direction with respect to beam portion 19. Axis A3 may be centered in the second widthwise direction with respect to the arm portions of receiver 10 and define an extension axis of longitudinal rod 6.

FIG. 5 illustrates an exploded parts view showing the integrally connected receiver 10 and side portion 20 separated from the set screw 4, rod 6, washer/c-ring 13, saddle 12, and pedicle screw 2. In the example illustration, side portion 20 includes a housing 21 that forms a hermetically sealed cavity therein for housing various microelectronics and sensors. Some example sensors may include a strain sensor (also referred to as a stress gauge), accelerometer, gyroscope, temperature gauge, and impedance sensor.

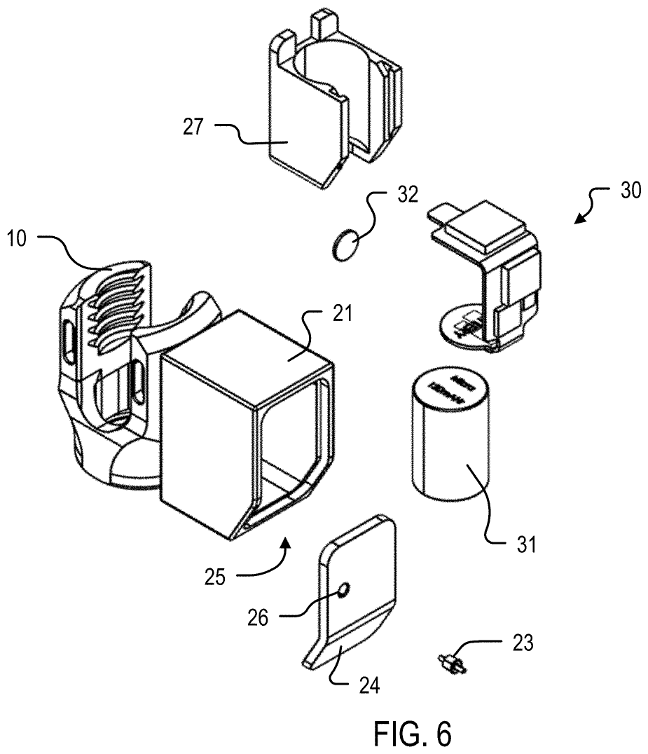

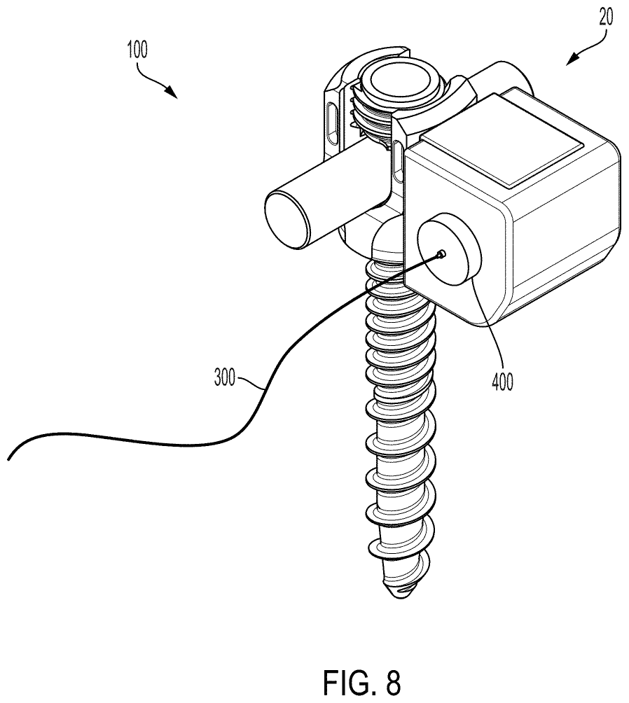

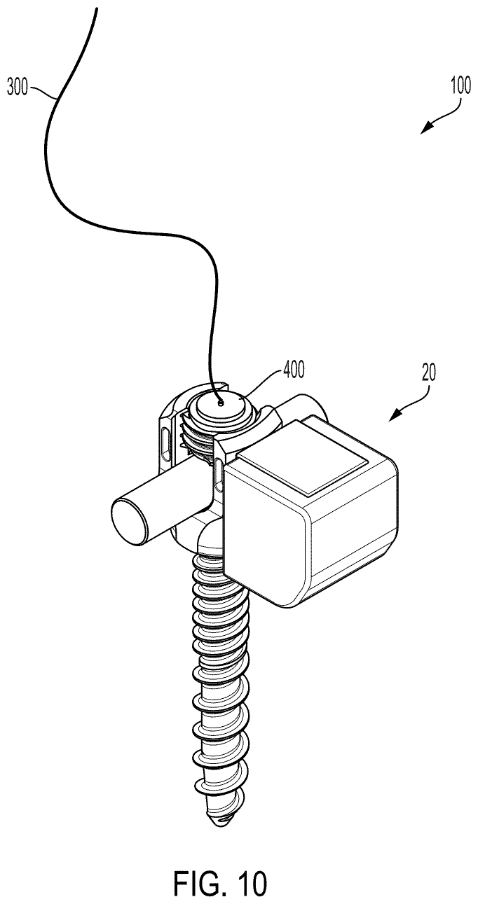

Referring to FIG. 6, housing 21 may define a cavity 25 therein for supporting various electronic components assembled in a microelectronics assembly 30 and a battery 31. In various embodiments, cavity 25 of housing 21 may be hermetically sealed such that the microelectronics assembly 30 and battery therein will not harm a patient when the system 100 is installed within the human body. The battery 31 and microelectronics assembly 30 may be installed within the cavity 25 in any suitable way. In the example embodiment, frame 27 may support the battery 31 and microelectronics assembly 30 securely within the cavity such that the microelectronics, battery 31, sensor 32 (e.g., strain gauge, temperature sensor), and antenna portion 300 (FIGS. 8, 10) are electrically connected. In the example embodiment, the microelectronics assembly 30 and battery 31 may be disposed inside of the cavity 25 and the cavity 25 may be sealed off by cover 24. Cover 24 may have a size and shape corresponding to an opening in housing 21 that exposes the cavity 25 therein. Due to the hermetically sealed nature of cavity 25, a feed-through connection 23 having suitable waterproof flanges may extend through an aperture 26 of cover 24. In this way, the feed-through connection 23 may be electrically connected to the microelectronics assembly 30 and the antenna portion 300 (FIG. 8) while ensuring that a hermetic seal of the electronics components is possible.

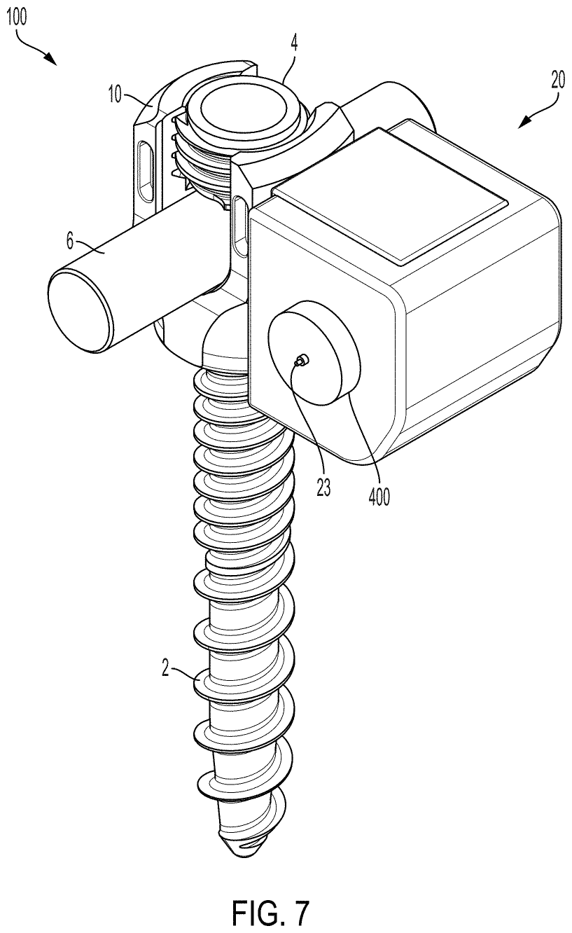

Referring to FIG. 7, housing 21 may be configured to receive an electronics cartridge 400. As illustrated, the cartridge is received in a horizontal direction, i.e., substantially parallel with the direction of the longitudinal rod 6. In other configurations, the cartridge may be substantially perpendicular to direction of the longitudinal rod 6. The electronics cartridge 400 may form a hermetically sealed cavity therein for housing electronics components and/or sensors similar to those disclosed above. Due to the hermetically sealed nature of the cartridge 400, a feed-through connection 23 having suitable waterproof flanges may extend through, e.g., the top surface of the cartridge to interface with the antenna 300. In this way, the feed-through connection 23 may be electrically connected to the microelectronics assembly 30 and to the antenna portion 300 (FIG. 8) while ensuring that a hermetic seal of the electronics components is possible. As illustrated, when the cartridge 400 is received within the housing 21, the top surface is nearly flush with the side of the housing 21. In some embodiments, electronics and/or battery 31 are housed in the cartridge 400, but one or more sensors are located outside of the cartridge (e.g., within the housing 21) and are electrically connected to the electronics and/or battery via a second feed-through connection 23 (not illustrated).

Referring to FIG. 8, the embodiment of FIG. 7 is shown with antenna portion 300 interfaced with feedthrough 23. As shown, the antenna 300 is a monopole or “whip” antenna made of a flexible material and is configured to be free floating. That is, the antenna 300 may extend away from the pedicle screw system 100 and into the surrounding tissue and be free to move with respect to the surrounding tissue. As disclosed above, the tissue surrounding the pedicle screw system 100, consisting primarily of muscle tissue, may tolerate a flexible antenna. The antenna 300 may extend toward the patient's skin, i.e., closer to an external reader. By extending closer to an external reader, the transmitted signal will have less tissue to pass through to reach the reader and, therefore, the signal will be less attenuated than a similar signal transmitted from a corresponding internal antenna of the pedicle screw system 100.

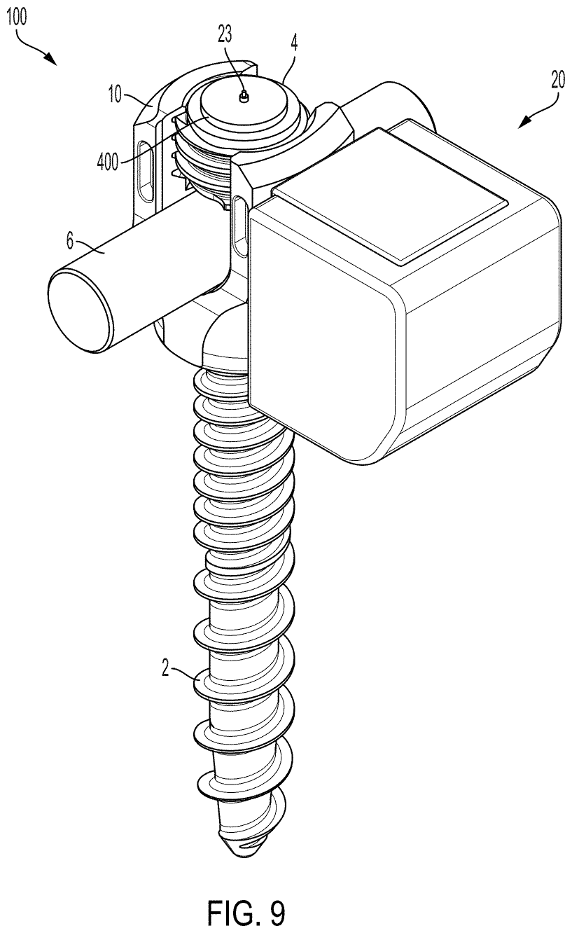

Referring to FIGS. 9 and 10, set screw 4 may be configured to receive the electronics cartridge 400. In some embodiments, the cartridge 400 is a removable modular design of standard dimensions such that it can be selectively installed within the set screw 4 of the pedicle screw system 100, received within the housing 21 of the pedicle screw system 100, mounted within a cage 1 of a digital spinal interbody implant system 200 (described below), or installed in any other spinal implant configured to receive the standard electronics cartridge 400. As in the embodiment illustrated in FIG. 8, the antenna 300 shown in FIG. 10, extends away from the feedthrough 23 of the pedicle screw system 100 and into surrounding tissue.

In some embodiments, the cartridge 400 includes a threaded exterior portion, such that the electronics cartridge 400 can be secured in place by screwing the cartridge 400 into the implant. In some embodiments, the cartridge 400 may be removably attached/retained using a cam lock, click-lock feature, or similar mechanical-linking feature configured to provide flexible/selectable integration. In some embodiments, the cartridge 400 may have unthreaded exterior surfaces and may simply be pressed into position. In some embodiments, the cartridge 400 is surrounded by one or more O-rings or other structure to enhance retention of the cartridge 400 within the implant. In some embodiments the cartridge 400 includes a battery of sufficient electrical storage capacity as to last for a typical patient-recovery period. In some embodiments, the battery may be rechargeable, e.g., inductively via the external reader, to extend the battery's service life.

Various antenna 300 and communication types may be, for example, MICS and BLE. As used herein, “MICS” may refer to the Medical Implant Communication System which may be a short-range communication technology that operates at a frequency from about 402 to 405 MHz. As used herein, “BLE” may refer to Bluetooth low energy communication standard. In some embodiments, the antenna may be a multi-band electrically coupled loop antenna (ECLA) antenna capable of operating in at least the MICS and LBE bands.

The microelectronics assembly 30 may have great variability in the types of circuitry and hardware due to the relatively large size of the side portion 20 and cavity 25. Example electronics components may include a (flexible) circuit board providing an electrical connection between the battery 31, sensor 32 (e.g., strain gauge, temperature sensor), and the various other electronics components. A non-limiting list of example electronics components may include a mainboard or other suitable printed circuit board (PCB), an application specific integrated circuit (ASIC), a micro controller, a wake-up sensor, a memory storage, a charge storage capacitor, and various mechanical electrical sensors or micro electromechanical systems (MEMs). Example MEMs may include a strain gauge, an impedance sensor, and/or a temperature sensor. However, other MEMs sensors may be incorporated in other embodiments depending on the particular use case.

In various embodiments the memory storage may be a non-transitory memory data store that may store information and/or data from various sensors and electronics components, for example. For example, one or more measurements of a sensor 32 (e.g., strain gauge, temperature sensor) may be stored in memory storage. As another example, a unique identifier associated with a load sensing assembly, a component thereof, or a set screw 4 may be stored in memory. One or more measurements received from sensor 32 may be used to make determinations of the condition of system 100 and/or treatment of a spinal disorder. For instance, proper placement of a longitudinal member 6, set screw 4 and/or pedicle screw 2 may result in an acceptable range of force measurements collected by a strain gauge. Measurements outside of this range may indicate a problem with the placement or positioning of the longitudinal member 6, set screw 4 and/or pedicle screw 2. For example, loosening of a critical component, construct failure, yield or fracture/breakage, improper torque, breakage of the bone segment or portion, the occurrence of fusion or amount of fusion, and/or the like.

In various embodiments, one or more measurements obtained by sensors 32 (e.g., strain gauge, temperature sensor) may be stored by an integrated circuit of a corresponding load sensing assembly such as, for example, in non-transitory computer readable memory storage disclosed above. In this way, the system 100 may be continuously powered by the battery 31 and obtain measurements over time. In some embodiments, the system 100 may “wake-up” at predetermined time periods to record various data points at predetermined time intervals. For example, the system 100 may be programmed to wake up at one-hour intervals, two hour intervals, etc. and record various data points to the memory storage. In this way, the power of the battery 31 may be preserved.

In various embodiments, an antenna 300 and/or wake up sensor may be interrogated by an external reader device (not illustrated) which may cause the transmission of data stored in the memory storage. In this embodiment, system 100 may not continuously transmit data stored in the memory storage, but rather may only transmit data stored in the memory storage when interrogated by a reader. For example, transmission of data may occur in response to being interrogated by the reader, or the transmission may be initiated at timed intervals. In various embodiments, the reader may receive the transmitted measurements, which may be displayed to a user such as a physician. Example readers may include at least one antenna for receiving and/or transmitting data across a suitable bandwidth and protocol similar to or the same as antenna 300. A reader may also include a central processing unit CPU, and a non-transitory computer readable medium (such as a memory unit or memory cell storing programmable computer implemented instructions).

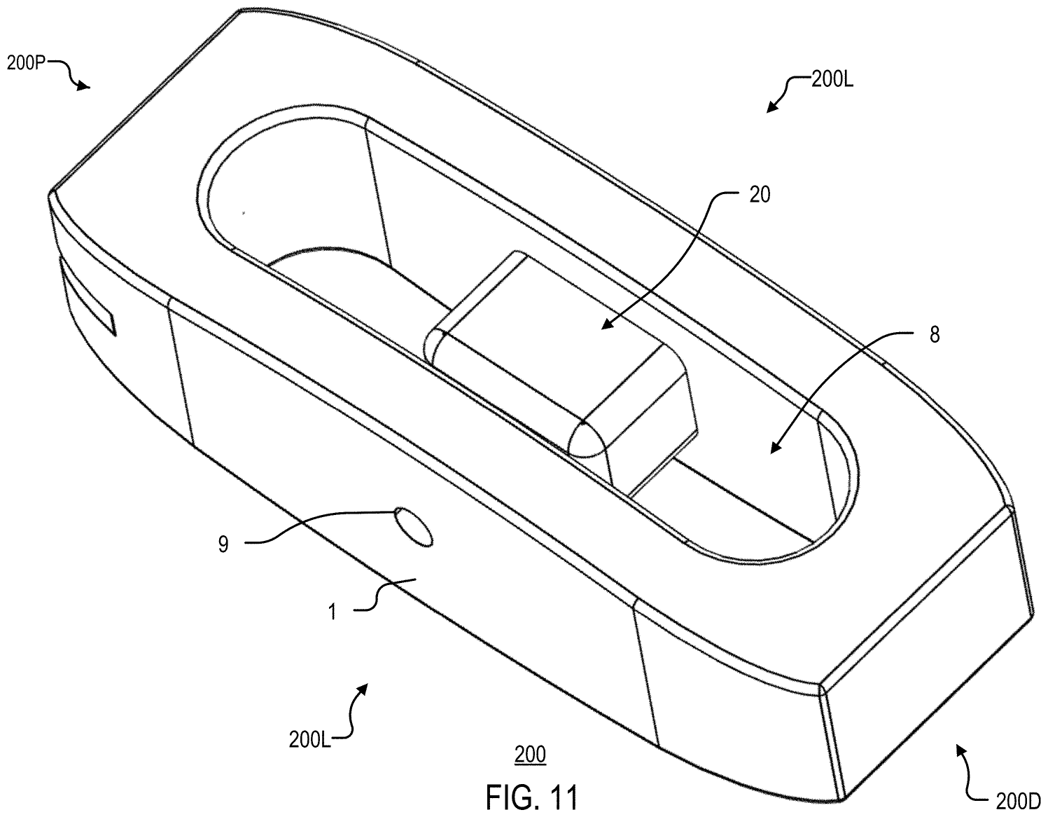

Referring generally to FIGS. 11-15, an example digital spinal interbody implant system 200 is disclosed. A system 200 may include an interbody cage 1 having a electronics portion 20 for supporting various electronic components and sensors therein as will be explained in further detail below. In various embodiments, interbody cage 1 may be integrally formed as a single monolithic component or interbody cage 1 may be an expandable cage with a superior endplate and an inferior endplate that may expand via an expansion mechanism. Cage 1 may extend in a longitudinal direction along from a proximal end 200P to a distal end 200D. In various embodiments, the proximal end 200P may have various features for grasping of the cage 1 to facilitate insertion and the distal end 200D may generally serve as the leading edge during the insertion of cage 1 into a patient as would be understood by a person of ordinary skill in the art. Additionally, cage 1 may extend in a widthwise direction from a first lateral end 200L to a second lateral end 200L. In various embodiments the cage 1 may include a graft window 8 and the electronics portion 20 may be disposed therein.

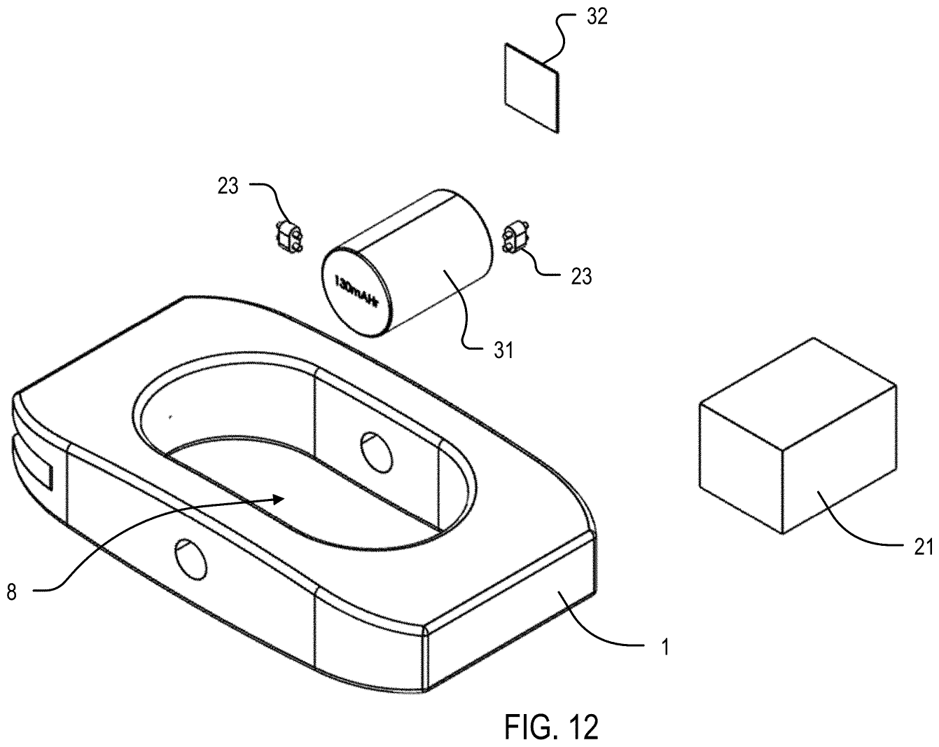

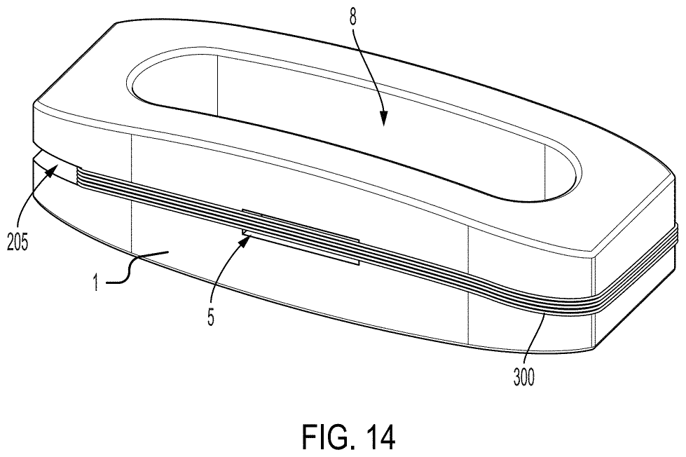

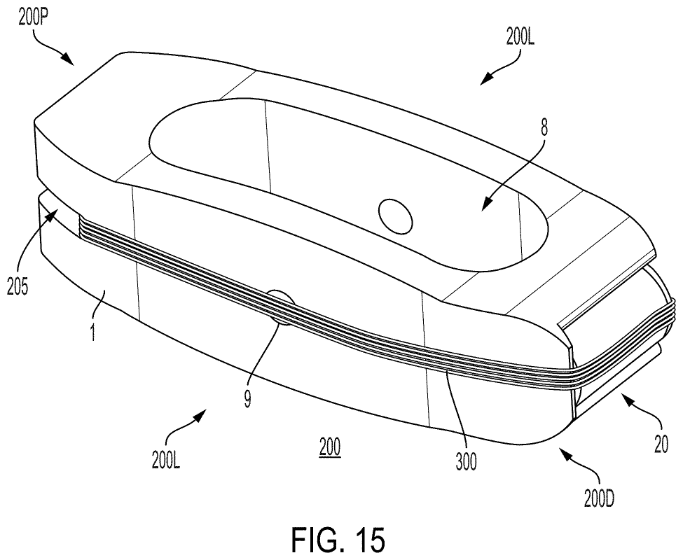

Referring to FIGS. 11-12 generally, various microelectronics may be disposed inside of the graft window 8 of cage 1. In this example embodiment, the electronics portion 20 may include an electronics housing 21. The electronics housing 21 may be located within graft window 8, on the exterior of cage 1 (e.g., attached to a side surface), or other suitable location. The electronics housing 21 may house any electronics componentry explained herein, e.g., battery 31, printed circuit boards, sensors, etc. In the example embodiment, a battery 31 is disposed inside of the electronics housing 21 and a first feed-through connection 23 may extend through electronics housing 21 and place the battery 31 and an antenna 300 (FIGS. 14, 15) in electrical connection. Additionally, a second feed-through connection 23 may extend through electronics housing 21 and place the battery 31 and a sensor 32 (e.g., strain gauge, temperature sensor) in electrical connection. Strain gauge 32 may be disposed anywhere on cage 1 to actively sense stress and strain applied to cage 1, e.g., by a superior vertebra and/or an inferior vertebra. For example, strain gauge 32 may be disposed on an interior sidewall of graft window 8 adjacent a medial portion of cage 1. In other embodiments, strain gauge 32 may be disposed on an interior sidewall of graft window 8 adjacent a proximal end 200P, a distal end 200D, or multiple strain gauges 32 may be disposed in any of the aforementioned relative locations. The electronics housing 21 may be affixed to cage 1 by any suitable means, e.g., a screw, pin, adhesive etc. In one embodiment, the electronics housing 21 and is placed inside of the graft window 8 and then surrounded by overmold to provide a hermetic seal to any component inside of the electronics housing 21. In at least one embodiment an impedance sensor protrudes into the graft window 8 for assessing the status of a fusion process and a temperature sensor is disposed inside of the cavity 25. In at least one embodiment, a temperature sensor is positioned to discern a body temperature of a patient in the region of the digital spinal interbody implant system 200, and in others a temperature sensor is positioned to discern a body temperature of a patient in a region adjacent a portion of digital pedicle screw system 100 that is directly exposed to patient tissue or contacting a portion of a longitudinal rod 6.

In some examples, the cage 1 may include a slotted aperture 5 (also referred to as a sensing slot 5) in a sidewall. In various embodiments, the slotted aperture may have a geometry, size, and location configured to transfer localized stress and strain to the strain gauge 32. Example electronics components may include a flexible circuit board providing an electrical connection between the battery 31, sensor 32 (e.g., strain gauge, temperature sensor), and the various other electronics components.

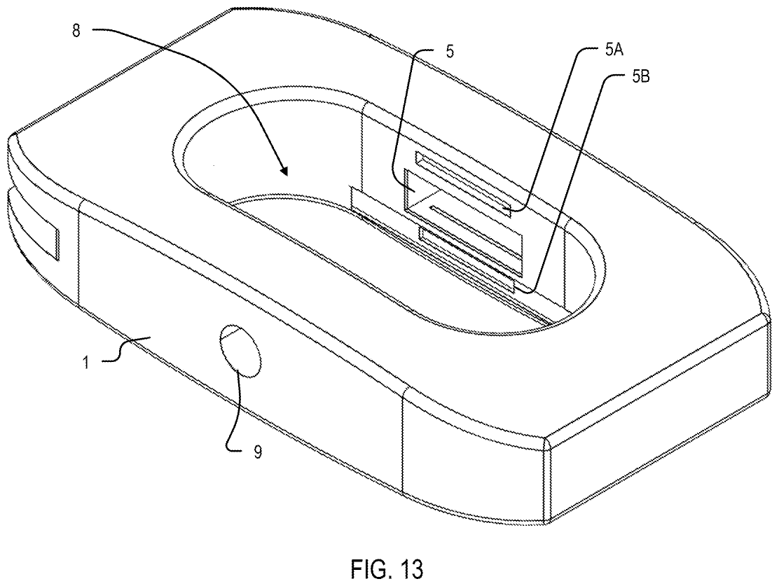

Referring to FIG. 13, implant system 200 may include a cage 1 having a fixation aperture 9 in a first sidewall and a primary sensing slot 5 (also referred to as a primary cavity) in a second sidewall. In the example embodiment, the fixation aperture 9 and sensing slot 5 each extend through a corresponding sidewall of cage 1 from the graft window 8 to the outside. Adjacent to the primary sensing slot 5, an upper secondary sensing slot 5A and a lower secondary sensing slot 5B are disposed adjacent to the primary sensing slot 5. The housing 21 may include a sensing protrusion having a size and shape corresponding to a size and shape of the primary sensing slot 5. Similarly, the housing 21 may include an upper secondary protrusion and a lower secondary protrusion having a size and shape corresponding to a size and shape of the upper secondary sensing slot 5A and lower secondary sensing slot 5B, respectively. This arrangement may be particularly advantageous at transmitting stress and strain experienced by cage 1 to the housing 21 and the sensing components therein may thereby have a heightened accuracy of detection.

In various embodiments, one or more measurements obtained by strain gauge 32 may be stored by an integrated circuit of a corresponding load sensing assembly such as, for example, in non-transitory computer readable memory as disclosed above. In turn, antenna 300 and/or electronics components 30 may be interrogated by a reader. For instance, an RFID chip may be read by an RFID reader. As another example, an NFC chip may be read by or may otherwise communicate with an NFC reader or other NFC-enabled device. In other embodiments, a custom protocol may be used, for example a 125 kHz inductive link. Example readers may include at least one antenna for receiving and/or transmitting data with antenna 300, a central processing unit CPU, and a non-transitory computer readable medium (such as a memory unit or memory cell storing programmable computer implemented instructions). In at least one embodiment, an electromagnetic reader (first reader) may transmit electromagnetic energy to the implant system 200 (or pedicle screw system 100) to power electronic components 30. An RFID reader or an NFC reader may be used separately to read, acquire, and/or interpret data received from antenna 300. A reader may interrogate an integrated circuit when in a certain proximity to the integrated circuit. In certain embodiments, a reader may interrogate an integrated circuit that has been implanted into a patient as part of an implant system 200 (or pedicle screw system 100), causing the integrated circuit to transmit one or more measurements to the reader. In other embodiments, an integrated circuit may communicate with a reader or other electronic device without being interrogated. The reader may receive the transmitted measurements, and may cause at least a portion of the measurements to be displayed to a user. For instance, a physician may use a reader to interrogate an RFID chip of a patient's implant. The reader may include a display, or may be in communication with a display device, which may display at least a portion of the measurements received from the RFID chip.

Referring to FIG. 14, the implant system 200 may include an external antenna 300. By locating the antenna 300 exterior to the implant system 200, the antenna may be made larger or may be located more closely to the patient's skin (and thus, to an external reader). In these ways, communication between the electronic components 30 and the external reader may be enhanced. As illustrated, the antenna 300 is wound around the exterior of the implant system 200. As disclosed above, the vicinity around an interbody cage includes, among other things, the spinal cord and associated nerves extending away from the spinal cord, blood vessels of various types, as so forth. Thus, the tissue surrounding the implant system 200 may be sensitive to interaction with a rigid antenna. Interaction between the antenna 300 and the surrounding tissue may be minimized by winding the antenna tightly around the implant system 200. In some embodiments, the implant system 200 includes a slot 205, or groove or other structure configured to maintain the position of the antenna 300. As illustrated in FIGS. 14 and 15, the antenna 300 may be wound around the exterior of the cage 1, and through slot 205.

Referring to FIG. 15, the implant system 200 may include one or more fixation apertures 9. In some embodiments, one or more fixation apertures 9 are configured to receive the modular electronics cartridge 400 described above. For example, the modular electronics cartridge 400 may be pressed (or screwed) into fixation aperture 9 such that the feedthrough 23 of the modular electronics cartridge 400 faces away from the implant system 200 (similar to the pedicle screw 100 discussed above) so that the feedthrough 23 can be electrically connected to the antenna 300. Because interbody cages are typically located further from the patient's skin than the head portion 3 of a pedicle screw, it may be more even important for the antenna 300 of the implant system 200 to be as close as possible to the patient's skin and/or to be substantial in size to facilitate communication with the external reader through a greater amount of tissue (and the accompanying attenuation). In some embodiments, the external antenna 300 is remote from the implant system 200. For example, the antenna 300 (or at least a substantial portion of the antenna 300) may be affixed to a nearby longitudinal rod 6, such that the antenna is even closer to the patient's skin (and the external reader).

Electronic components 30 may include a passive integrated circuit. An example passive integrated circuit may refer to an arrangement where electronic components 30 do not include an internal power source. For example, electronic components 30 may be powered by energy transmitted from a reader. With respect to electronic components 30 having a passive integrated circuit, the passive integrated circuit may not transmit information until interrogated by a reader. For example, a reader may transmit electromagnetic energy directed at the passive integrated circuit to wirelessly power the passive integrated circuit. At least two advantages of using an integrated circuit that does not include a battery or require a battery is reliability, and reduction in space within the cavity that houses the electronic components 30 forming a passive integrated circuit.

In various embodiments, one or more sensors of electronic components 30 may transmit information by directly modulating a reflected signal, such as an RF signal. The strain gauge (or other) sensors 32 may form a Wireless Passive Sensor Network (WPSN), which may utilize modulated backscattering (MB) as a communication technique. External power sources, such as, for example, an RF reader or other reader, may supply a WPSN with energy. The sensor(s) of the WPSN may transmit data by modulating the incident signal from a power source by switching its antenna impedance.

In another embodiment, an integrated circuit may be active, meaning that the chip is battery-powered and capable of broadcasting its own signal. An active integrated circuit may transmit information in response to be interrogated by a reader, but also on its own without being interrogated. For instance, an active integrated circuit may broadcast a signal that contains certain information such as, for example, one or more measurements gathered by an associated strain gauge. An active integrated circuit may continuously broadcast a signal, or it may periodically broadcast a signal. Power may come from any number of sources, including, for example, thin film batteries with or without encapsulation or piezo electronics.

One or more measurements received from a load sensing assembly may be used to make determinations of the condition of a spinal implant and/or treatment of a spinal disorder. For instance, proper placement of a longitudinal member 6, set screw 4 and/or anchoring member may result in an acceptable range of force measurements collected by a strain gauge of a load sensing assembly. Measurements outside of this range may indicate a problem with the placement or positioning of a longitudinal member, set screw and/or anchoring member such as, for example, loosening of a set screw 4 and/or anchoring member, longitudinal member failure, construct failure, yield or fracture/breakage, improper torque, breakage of the bone segment or portion, the occurrence of fusion or amount of fusion, and/or the like. In these instances, the reader may contain a range of pre-determined acceptable values corresponding to the strain gauge and/or other MEMs sensors 32. If the actual measured reading of the strain gauge and/or other MEMs sensors 32 falls outside of the range, the reader may notify an end user, a hospital management system, and/or the patient. For example, a patient may continuously or regularly monitor the actual measured readings of an implant system 200 (or pedicle screw system 100) on an outpatient basis with a reader. In some embodiments, a reader may be configured to relay information received from antenna 300 to a secondary processing component such as an external display, computer, server, hospital management system, or other type of data processing equipment. The secondary processing component may process information received by the reader from antenna 300 via a processor, controller, and memory configured to execute programmable computer implemented instructions. In this way, disclosed systems increase the likelihood that a patient can detect a malfunction, such as loosening of a set screw 4, before catastrophic failure.

One or more tools or instruments may include a reader which may be used to gather information from one or more integrated circuits of electronic components 30 during or in connection with a procedure. For instance, a torque tool (not illustrated) may be used to loosen or tighten the set screw 4. A torque tool may include a reader, or may be in communication with a reader, such that a user of the torque tool is able to obtain, in substantially real time, one or more measurements relating to the set screw 4 and longitudinal rod 6 placement that are measured by a strain gauge 32 of a load sensing assembly of the set screw 4 via the tool. For instance, as a user is applying torque to a set screw 4, the user may see one or more force measurements between the set screw 4 and the longitudinal member 6 in order to determine that the positioning of the set screw 4 and/or longitudinal member 6 is correct and that the proper force is being maintained. In certain embodiments, a tool or instrument may include a display device (not illustrated) on which one or more measurements may be displayed. In other embodiments, a tool or instrument may be in communication with a display device (not illustrated), and may transmit one or more measurements for display on the display device via a communications network.

In some embodiments, an electronic device, such as a reader or an electronic device in communication with a reader (not illustrated), may compare one or more measurements obtained from an integrated circuit to one or more acceptable value ranges. If one or more of the measurements are outside of an applicable value range, the electronic device may cause a notification to be made. For instance, an electronic device may generate an alert for a user, and cause the alert to be displayed to the user via a display device. Additionally or alternatively, an electronic device may send an alert to a user such as via an email message, a text message, a notification, or otherwise.

An integrated circuit of electronics components 30 may store a unique identifier associated with the components to which the load sensing assembly corresponds. For example, an integrated circuit of electronics components 30 for an implant system 200 (or pedicle screw system 100) may store a unique identifier associated with the implant system 200 (or pedicle screw system 100). For example, when a reader interrogates an integrated circuit, the integrated circuit may transmit a unique identifier for a component that is stored by the integrated circuit to the reader. Having access to a unique identifier for a component may help a user ascertain whether the measurements that are being obtained are associated with the component of interest. Also, having access to a unique identifier for a component may help a user take inventory of one or more components. For instance, after spinal surgery, a physician or other health care professional may use a reader to confirm that all of the implant components allocated for the procedure have been used and are positioned in a patient. This may also help with the detection and verification of screws in a patient's body.

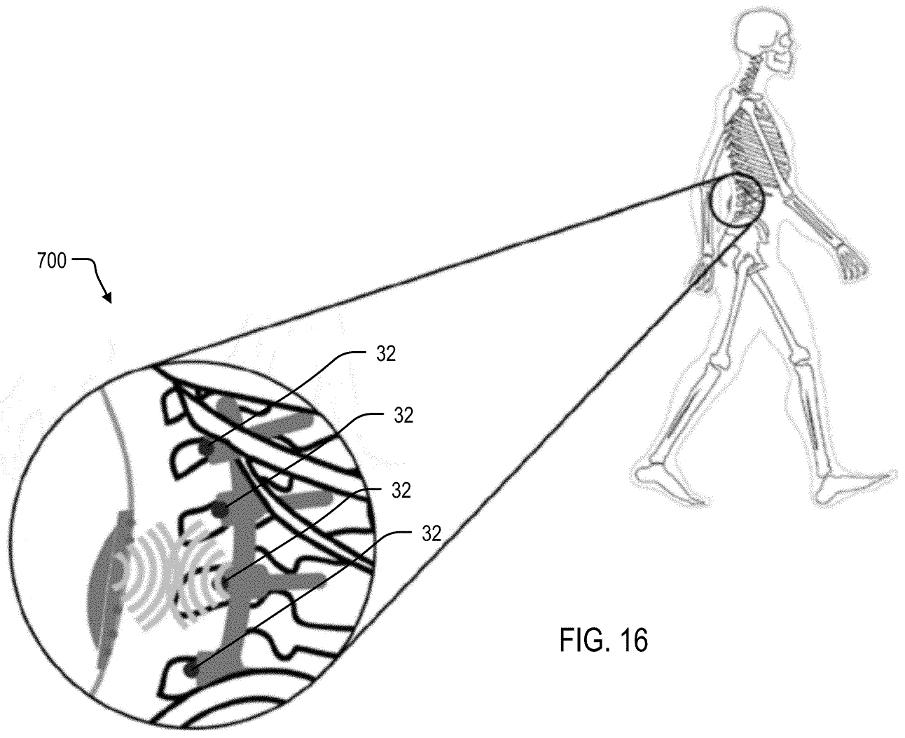

FIG. 16 illustrates an example of a surgical site (SS) monitoring system 700 that may utilize example digital pedicle screw systems 100 and/or digital spinal interbody implant system 200 disclosed herein. In some embodiments, the SS monitoring system 700 may be a surgical site load monitoring system (using one or more strain gauges 32) and/or an infection monitoring system (using one or more temperature sensors 32). In at least one embodiment, a temperature sensor is positioned to discern a body temperature of a patient in the region of the digital spinal interbody implant system 200, and in others a temperature sensor is positioned to discern a body temperature of a patient in a region adjacent a portion of digital pedicle screw system 100 that is directly exposed to patient tissue or contacting a portion of a longitudinal rod 6. FIG. 16 illustrates a single implant system (e.g., a single spinal-fusion construct) having multiple separate sensor-equipped implants, each of which may have one or more sensors. Other embodiments within the scope of this disclosure include a single sensor-equipped implant having one or more sensors. Still other embodiments include multiple implant systems, e.g., multiple spinal-fusion constructs, or one spinal-fusion construct and a separate sensor-equipped implant. Other combinations and permutations of implant systems and/or sensor-equipped implants are also within the scope of the disclosure.

In one or more embodiments, the SS monitoring system 700 may include an array of implants, in which one or more of the implants have any type of MEMs sensor 32 as previously disclosed. For the cases in which the SS monitoring system 700 includes an array of implants having various MEMs sensors 32, the received data from the one or more MEMs sensors 32 may be compared to one another to diagnose the quality of the surgical procedure, the integrity of the implant, and/or an infection at the surgical site.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

The breadth and scope of this disclosure should not be limited by any of the above-described example embodiments, but should be defined only in accordance with the following claims and their equivalents.

What is claimed is:

1. A spinal implant comprising:
an electronics portion including a housing defining a sealed cavity for supporting an electronics assembly and a battery therein;
at least one antenna in electrical communication with the electronics assembly; and
at least one sensor in electrical communication with the electronics assembly, wherein the at least one antenna is located external to the housing and is configured to transmit information received from the at least one sensor to an external device.

2. The spinal implant of claim 1, wherein:
the housing comprises a hermetically sealed, removable electronics cartridge having at least one antenna interface; and
the electronics portion is configured to receive the removable electronics cartridge.

3. The spinal implant of claim 2, wherein the removable electronics cartridge has a threaded exterior that mates with the electronics portion.

4. The spinal implant of claim 2, wherein the at least one sensor is located within the removable electronics cartridge.

5. The spinal implant of claim 1, wherein:
the spinal implant comprises a pedicle screw including a set screw;
the housing comprises a removable electronics cartridge having at least one antenna interface; and
the set screw is configured to receive the removable electronics cartridge.

6. The spinal implant of claim 1, wherein:
the spinal implant comprises an interbody cage; and
the at least one antenna is wound around an exterior surface of the interbody cage.

7. The spinal implant of claim 6, wherein:
the interbody cage includes a slot; and
the at least one antenna is wound around the exterior surface of the interbody cage and through the slot.

8. The spinal implant of claim 1, wherein:
the spinal implant comprises a pedicle screw; and
the at least one antenna is configured to flexibly extend away from the pedicle screw.

9. The spinal implant of claim 1, wherein the at least one sensor comprises at least one temperature sensor configured to measure a temperature of a surgical site.

10. The spinal implant of claim 1, wherein the at least one sensor comprises at least one strain gauge configured to measure a localized force experienced by the spinal implant.

11. The spinal implant of claim 1, wherein:
the spinal implant comprises an interbody cage including a graft window; and
the at least one sensor comprises at least one impedance sensor located within the graft window and configured to measure a status of a fusion process.

12. The spinal implant of claim 1, wherein the at least one sensor is chosen from the group comprising: accelerometer, gyroscope, strain gauge, pressure sensor, pH sensor, impedance sensor, optical sensor, and temperature sensor.

13. The spinal implant of claim 1, wherein the battery is configured to be inductively recharged.

14. A surgical site monitoring system comprising:
a first spinal implant comprising:
a first electronics portion including a first housing defining a first sealed cavity for supporting a first electronics assembly and a first battery therein;
at least one first antenna in electrical communication with the first electronics assembly; and
at least one first sensor in electrical communication with the first electronics assembly, wherein the at least one first antenna is located external to the first housing and is configured to transmit first information received from the at least one first sensor to an external device; and
a second spinal implant comprising:
a second electronics portion including a second housing defining a second sealed cavity for supporting a second electronics assembly and a second battery therein;
at least one second antenna in electrical communication with the second electronics assembly; and
at least one second sensor in electrical communication with the second electronics assembly, wherein the at least one second antenna is located external to the second housing and is configured to transmit second information received from the at least one second sensor to the external device.

15. The surgical site monitoring system of claim 14, wherein:
the first and second housings comprise hermetically sealed, removable electronics cartridges having at least one antenna interface; and
the first and second electronics portions are configured to receive the removable electronics cartridges.

16. The surgical site monitoring system of claim 14, wherein the at least one first sensor and the at least one second sensor comprise at least one temperature sensor configured to collect temperature measurements within a patient.

17. The surgical site monitoring system of claim 14, wherein the at least one first sensor and the at least one second sensor comprise at least one impedance sensor configured to measure a status of a fusion process.

18. The surgical site monitoring system of claim 14, wherein the first and second spinal implants each comprises a pedicle screw or an interbody cage.

19. The surgical site monitoring system of claim 14, wherein the first and second antennas are each configured to flexibly extend away from the first and second spinal implants, respectively.

20. The surgical site monitoring system of claim 14, wherein at least one of the first battery and the second battery are configured to be inductively recharged.
